(19) United States
(12) Patent Application Publication
(10) Pub. No.: US 2012/0316388 A1
(43) Pub. Date: Dec. 13, 2012

(54) Adult Toy

(76) Inventor: Chia-Yen Lin, Dongguan City (CN)

(21) Appl. No.: 13/158,370

(22) Filed: Jun. 10, 2011

(51) Int. Cl. A61F 5/00

(52) U.S. Cl. 600/38

(57) ABSTRACT

An adult toy includes an elongated elastic body which has an inner space defined therein and an opening defined at one end thereof. A sealing device or a one-way device is disposed at the opening to seal the inner space. The user can make fluid flow through the sealing device or the one-way device into the inner space to inflate the elastic body, or release the fluid out of the inner space to deflate the elastic body. When the fluid is completely released, the adult toy is greatly reduced in size and is easy to store or carry. The elastic body may connect with a reservoir. The fluid received in reservoir can be forced to enter the elastic body and the fluid received in the elastic body can be released to the reservoir, thereby adjusting the stiffness and the size of the elastic body.

### BACKGROUND OF THE INVENTION

1. Field of the Invention

The present invention relates to an adult toy, and more particularly, to an adult toy having adjustable stiffness, diameter and length.

2. The Prior Arts

Sex is a normal demand for men and women. Some people engage sexual behaviors with sex workers. However, it is illegal in some areas and has a high risk to get sexual transmitted diseases, such as gonorrhea, syphilis and AIDS. Therefore, in recent days, masturbation is recognized as an option for people to avoid STD infection. The stimulations can be performed manually or by use of objects, so that adult toys are increasingly popular in the market. The adult toys not only are welcomed by the single, marriage counselors and physicians also encourage their use as an adjunct to a healthy and meaningful relationship.

The adult toys are designed for males and females. The adult toys for females include dildos and vibrators. The dildos may be made of various materials, such as glass, metal and PVC. Recently, the dildos are generally made of silicone rubber and made to look and feel like a penis. The vibrator includes a vibrating unit to stimulate the nerves for a relaxing and pleasurable feeling.

The conventional adult toys for females have fixed size and stiffness so that the users cannot change the size and stiffness according to their preference. Furthermore, they occupy a certain space and therefore it is inconvenient to carry when travel. Moreover, when the luggage goes through the X-ray machine, the adult toys would be seen by the security workers and the personal privacy is breached.

### SUMMARY OF THE INVENTION

A primary objective of the present invention is to provide an adult toy whose size and stiffness can be adjusted according to personal preference. Another objective of the present invention is to provide an adult toy that is easy to carry when deflated.

In order to achieve the objectives mentioned above, an adult toy according to the present invention comprises an elongated elastic body and a sealing device having a path. The elastic body is made of a resilient material capable of changing size. The elastic body has an inner space defined therein and an opening defined at an end thereof. The sealing device is disposed in the opening of the elastic body to seal the inner space. The user may inject fluid into or withdraw the fluid out of the inner space of the elastic body through the path. Thus, the amount and pressure of the fluid in the inner space would change the size and stiffness of the elastic body, respectively.

The sealing device may be an elastic plug. When there is no external force, the path is closed due to the characteristics of the resilient material of the sealing device. Thus, the fluid in the inner space would not leak out of the inner space. When an injection needle is inserted to pass through the path and enter the inner space, the fluid can be injected into or withdrawn from the inner space of the elastic body.

According to another embodiment of the present invention, a reservoir is connected to the opening of the elastic body and a one-way device is disposed between the elastic body and the reservoir. The user can operate the adult toy and make fluid flow from the reservoir into the inner space through the one-way device. The one-way device would prevent the fluid from flowing back to the reservoir. The user may also operate the one-way device to release the fluid from the inner space into the reservoir through the one-way device, so that the length, diameter and stiffness of the elastic body can be adjusted by the pressure in the inner space of the elastic body.

The reservoir is a receptacle to receive the fluid. The reservoir may be a resilient reservoir with a hole (an open reservoir) or without a hole (an enclosed reservoir). The reservoir may also be a rigid and enclosed reservoir.

The one-way device is disposed between the elastic body and the reservoir to seal the opening of the elastic body. The one-way device is provided with a switch that is operable to allow the fluid to flow out of the inner space and into the reservoir.

The one-way device may be a one-way valve having a switch.

The one-way device may also include an electric pump and a tube connected with the pump and extended to an inside of the reservoir. The users can operate the pump to send fluid from the reservoir into the elastic body to increase the pressure in the elastic body. The users can also operate the switch of the pump allowing the fluid in the elastic body back to the reservoir to reduce the pressure in the elastic body, thereby adjusting the length, the diameter and the stiffness of the elastic body.

The reservoir may be integrally formed with the elastic body. The reservoir and the elastic body may also be individually manufactured and then connected to each other.

The elastic body of the adult toy may further include a plurality of bosses extending from the outer surface thereof so as to increase stimulation within the user's body.

The elastic body and the reservoir are shaped as the penis and the scrotum, respectively.

### BRIEF DESCRIPTION OF THE DRAWINGS

The present invention will be apparent to those skilled in the art by reading the following detailed description of preferred embodiments thereof, with reference to the attached drawings, in which:

FIG. 1 is a cross sectional view showing an adult toy according to a first embodiment of the present invention before fluid is filled in;

FIG. 2 shows that an injection needle injects fluid into the adult toy of FIG. 1;

FIG. 3 is a cross sectional view showing an adult toy according to a second embodiment of the present invention including an elastic body, a reservoir integrally formed with the elastic body, and a one-way device disposed between the elastic body and the reservoir;

FIG. 4 is a cross sectional view showing that the elastic body may further have a plurality of bosses extending from the outer surface thereof;

FIG. 5 is a cross sectional view showing that the reservoir is squeezed to force the fluid to flow from the reservoir into the elastic body;

FIG. 6 is a cross sectional view showing that the reservoir having a hole is dipped in water and the water is introduced into the reservoir through the hole;

FIG. 7 is a cross sectional view showing an adult toy according to a third embodiment of the present invention, wherein the elastic body and the reservoir are two individual parts and a one-way valve is connected to an opening of the elastic body;

FIG. 8 is a cross sectional view showing an adult toy according to a fourth embodiment of the present invention, wherein the elastic body and the reservoir are two individual parts and the one-way valve is connected to an opening of the reservoir;

FIG. 9 is a cross sectional view showing an adult toy according to a fifth embodiment of the present invention, wherein a pump is connected to the elastic body and the reservoir is connected to the elastic body; and

FIG. 10 shows that the elastic body and the reservoir are two individual parts which can be connected to each other or separated from each other; the pump is connected to the elastic body.

### DETAILED DESCRIPTION OF THE PREFERRED EMBODIMENT

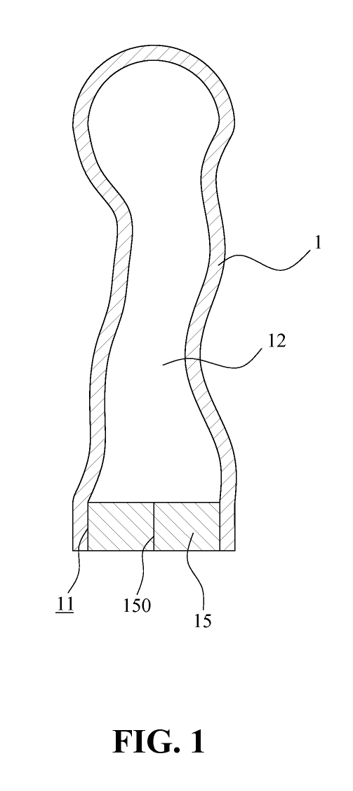

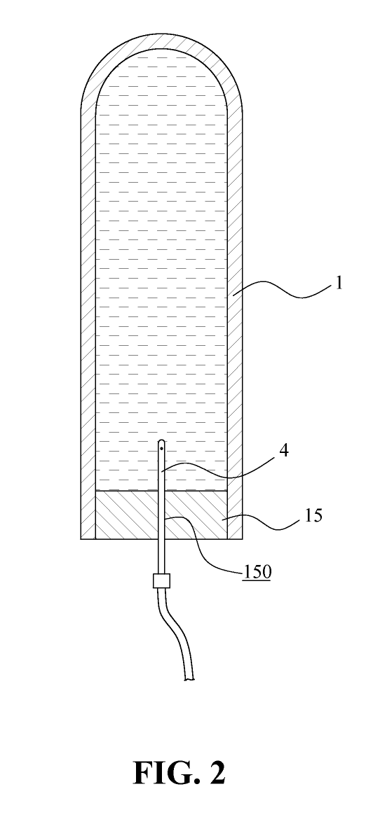

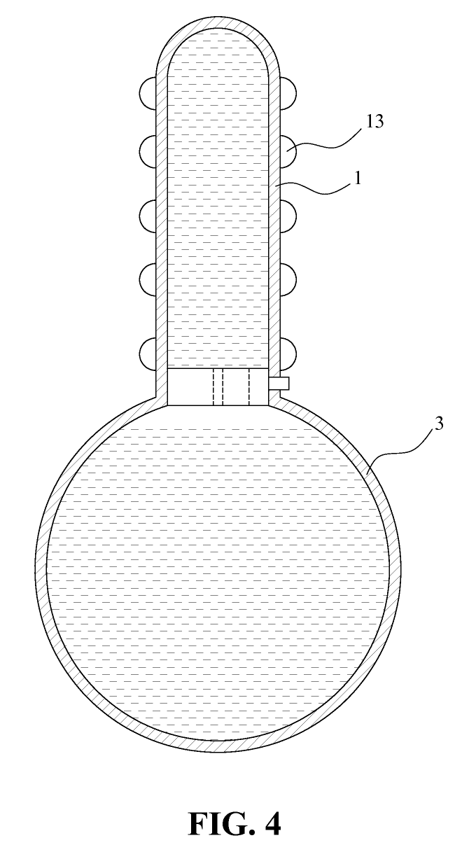

Referring to FIG. 1, an adult toy according to a first embodiment of the present invention comprises an elongated elastic body 1 and a sealing device 15. The elastic body 1 is made of a non-toxic resilient material, such as PU and silica gel. The elastic body 1 has an inner space 12 defined therein. A first end of the elastic body 1 includes an opening 11 and a second end of the elastic body 1 is a closed end. The opening 11 communicates with the inner space 12. The elastic body 1 may further includes a plurality of integrally-formed bosses 13 extending from an outer surface thereof as shown in FIG. 4. The sealing device 15 is an elastic plug, such as a rubber plug, and is disposed at the opening 11 to seal the inner space 12. The sealing device 15 includes a path 150. When there is no force applied to the path 150, the path 150 is automatically closed due to the characteristics of the resilient material. Thus, the sealing device 15 can prevent fluid (liquid or gas) from leaking out of the inner space 12. As shown in FIG. 2, an injection needle 4 connected to a syringe or other pumping device (not shown in drawings) is inserted through the path 150 to inject fluid into the inner space 12 of the elastic body 1, thereby inflating the elastic body 1. Thus, increasing the amount of the fluid in the inner space 12 can lengthen and enlarge the elastic body 1 and increasing the fluid pressure in the inner space 12 can stiffen the elastic body 1. Similarly, the fluid can be release out of the inner space 12 and therefore the elastic body 1 is deflated. The user can fill the fluid into or release the fluid out of the inner space 12 to customize the length, the diameter and the stiffness of the elastic body 1 according to personal preference.

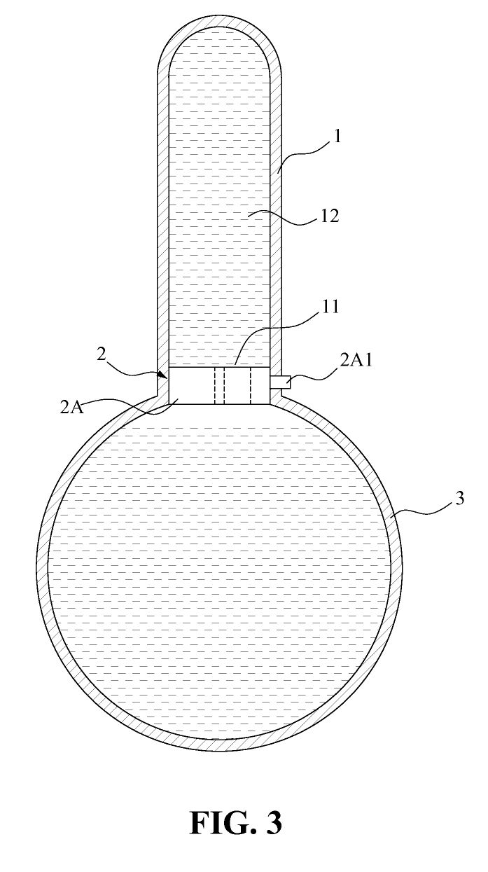

Referring to FIG. 3, an adult toy according to a second embodiment of the present invention includes an elongated elastic body 1, a one-way device 2 and a reservoir 3. The elastic body 1 is made of a non-toxic resilient material, such as PU and silica gel, and has an inner space 12 defined therein. An opening 11 is defined at a first end of the elastic body 1 and a second end of the elastic body 1 is a closed end. The opening 11 communicates with the inner space 12. The elastic body 1 may further include a plurality of integrally-formed bosses 13 extending from an outer surface thereof as shown in FIG. 4. The one-way device 2 comprises a one-way valve 2A having a one-way switch 2A1. The one-way valve 2A is disposed at the opening 11 of the elastic body 1 so as to seal the inner space 12. The one-way valve 2A allows fluid to enter the inner space 12 but the fluid in the inner space 12 cannot flow out from the one-way valve 2A unless the users operate the switch 2A1 to release the fluid from the inner space 12. The reservoir 3 receives liquid or gas therein and is connected to the opening 11 of the elastic body 1. The reservoir 3 may be made of the same resilient material as the elastic body 1 and integrally formed with the elastic body 1. The adult toy according to the present invention may simulate the shape of the man's members. The elastic body 1 and the reservoir 3 are shaped as a penis and a scrotum, respectively. As shown in FIG. 3, the reservoir 3 is an enclosed reservoir and water, oil, or air is received therein.

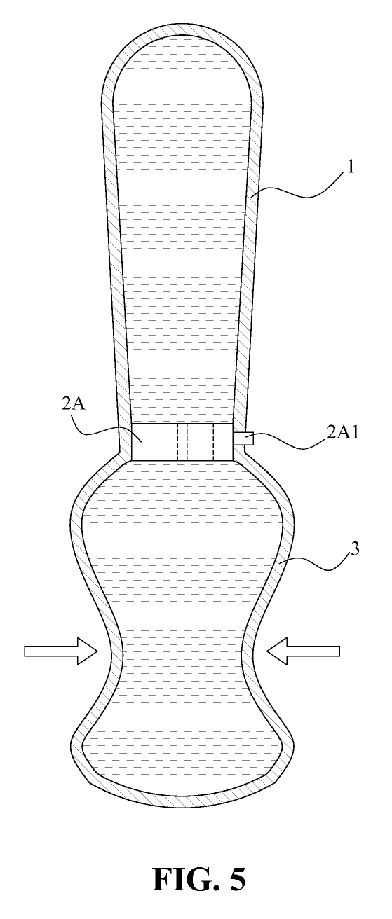

As shown in FIG. 5, the resilient reservoir 3 is squeezed to force the fluid received in the reservoir 3 to flow through the one-way valve 2A and enter the elastic body 1. The fluid would inflate the elastic body 1, so that the length, the diameter and the stiffness of the elastic body 1 would increase until it just like an erect penis. On the other way, the switch 2A1 is operated to make the fluid in the elastic body 1 flow back to the reservoir 3 through the one-way valve 2A to reduce the amount and pressure of the fluid in the elastic body 1. The length, the diameter and the stiffness of the elastic body 1 would decrease. Therefore, the user can customize the size and the stiffness of the elastic body 1 according to personal preference.

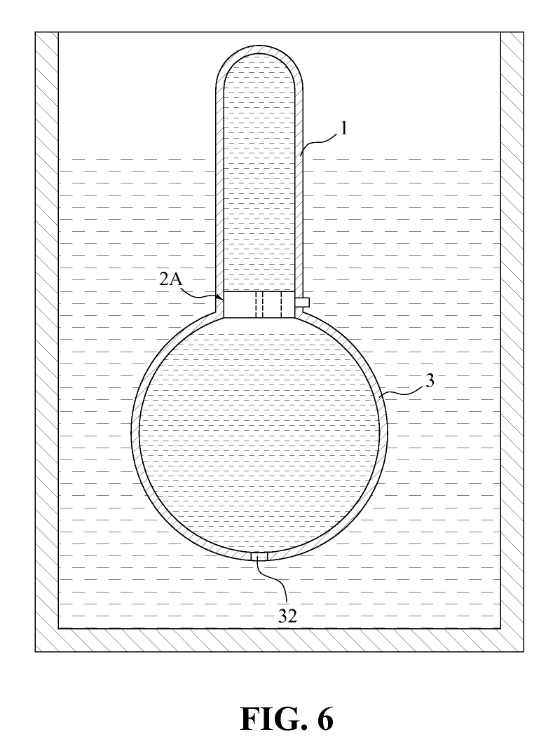

As shown in FIG. 6, the reservoir 3 may further have a hole 32. If the user prefers the elastic body 1 to be warm, the user can dip the adult toy into warm water, or at least immerse the reservoir 3 into the warm water. After the warm water enters the reservoir 3 through the hole 32, the hole 32 is sealed and then the warm water can be forced into the inner space 12 of the elastic body 1. A diameter of the hole 32 is smaller than that of a path (not shown) of the one-way valve 2A, so that when the reservoir 3 is squeezed, even if the hole 32 is not sealed, the warm water would pass through the one-way valve 2A and enter the elastic body 1 due to the smaller pressure. Thus, the elastic body 1 is inflated and the length, the diameter and the stiffness of the elastic body 1 increase. The user can also operate the switch 2A1 to releases the warm water received in the elastic body 1 back to the reservoir 3 through the one-way valve 2A, thereby adjusting the size and the stiffness of the elastic body 1. When the reservoir 3 is located in the atmosphere instead of being immersed in the water, the air enters the reservoir 3 through the hole 32. Therefore, no matter the hole 32 is sealed or not, when the reservoir 3 is squeezed, the air in the reservoir 3 is forced to pass through the one-way valve 2A and enters the elastic body 1, so that the length, the diameter and the stiffness of the elastic body 1 are increased. The users may also releases the air received in the elastic body 1 back to the reservoir 3 through the one-way valve 2A by operating the switch 2A1. Thus, the size and the stiffness of the elastic body 1 are customized by the user.

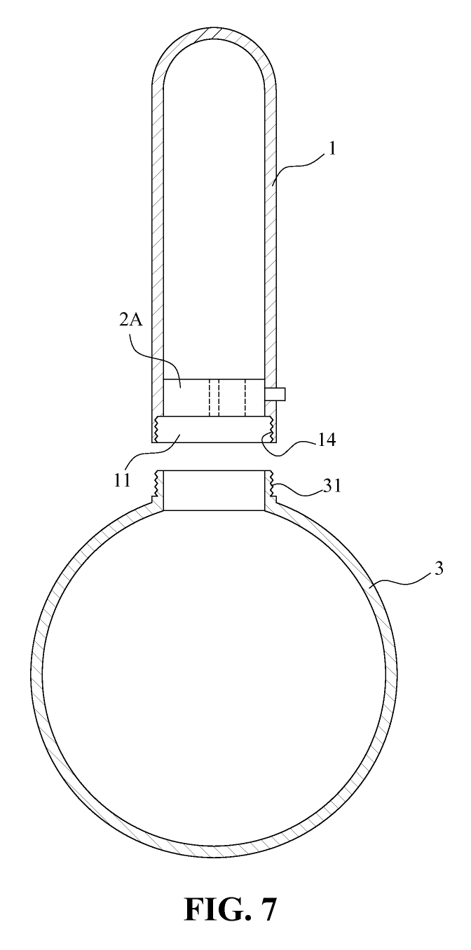

FIG. 7 shows an adult toy according to a third embodiment of the present invention. The difference between the third embodiment and the previous embodiments is that the elastic body 1 and the reservoir 3 are two individual parts which can be connected with each other or disconnected from each other. The one-way valve 2A is disposed at the opening 11 of the elastic body 1 and inner threads 14 are defined in an inside of the opening 11. An opening of the reservoir 3 has outer threads 31 which are threadedly connected with the inner threads 14, thereby connecting the elastic body 1 with the reservoir 3.

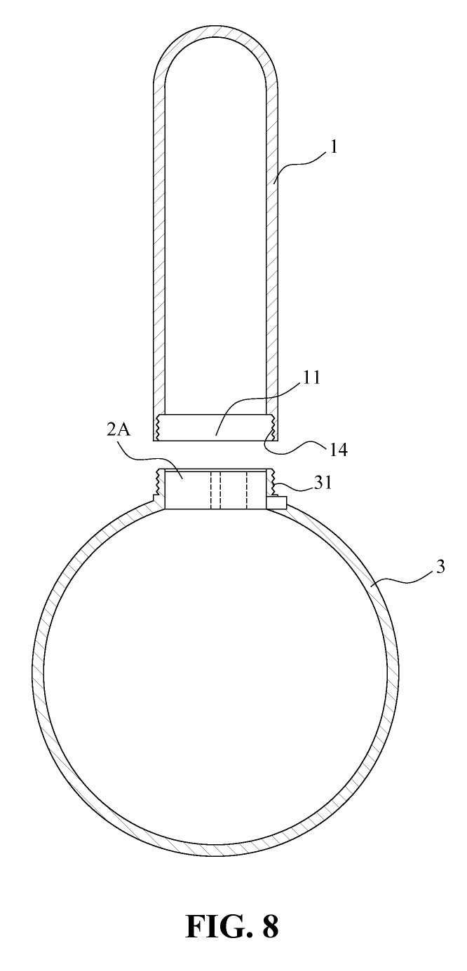

FIG. 8 shows an adult toy according to a fourth embodiment of the present invention. The elastic body 1 and the reservoir 3 are individually manufactured and then connected to each other. The opening 11 of the elastic body 1 includes inner threads 14 and the opening of the reservoir 3 has the one-way valve 2A disposed therein. The opening of the reservoir 3 has outer threads 31 to threadedly connect with the inner threads 14 of the elastic body 1, thereby detachably connecting the elastic body 1 with the reservoir 3.

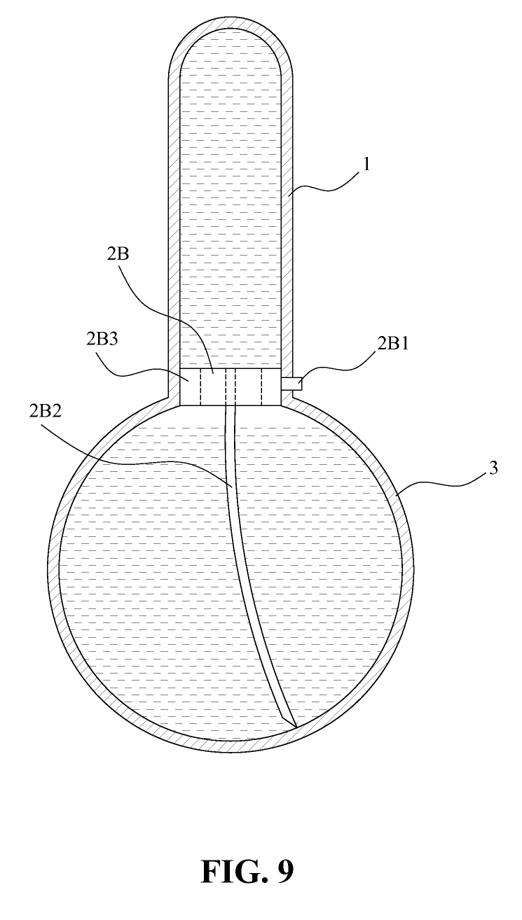

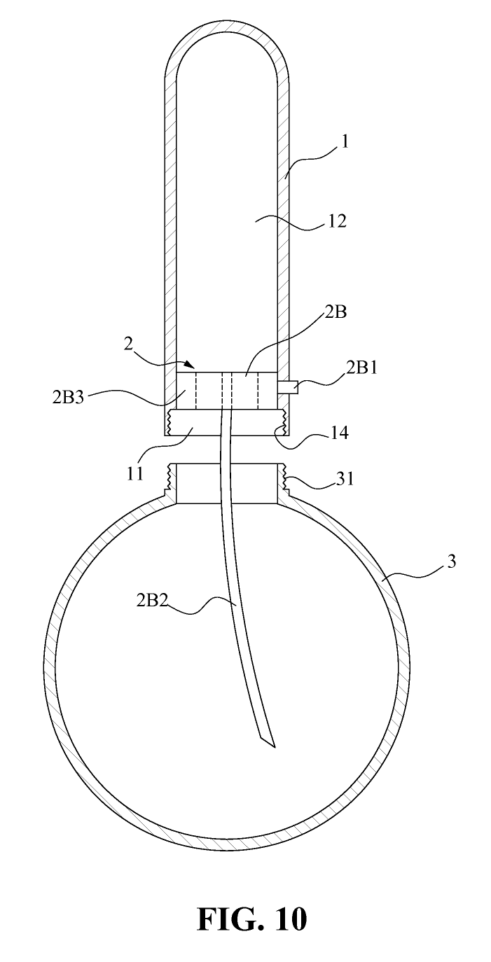

FIG. 9 shows an adult toy according to a fifth embodiment of the present invention. The one-way device 2 includes an electric one-way pump 2B and a switch 2B1. The pump 2B is electrically connected to a power source 2B3. A tube 2B2 is connected to the pump 2B and extends to an inside of the reservoir 3. The power source 2B3 is preferred to be a battery unit using direct current. The power source 2B3 may also be an alternative-current power source which is cooperated with an adapter to provide power to the pump 2B. When the one-way device 2 includes the pump 2B, the reservoir 3 may be made of a resilient material as previous embodiments or a non-resilient material (rigid reservoir). The reservoir 3 may be integrally formed with the elastic body 1 as shown in FIG. 9. The reservoir 3 and the elastic body 1 may also be individually manufactured and then detachably connected to each other as shown in FIG. 10. When the reservoir 3 and the elastic body 1 are individually manufactured, the opening 11 of the elastic body 1 has inner threads 14 and the opening of the reservoir 3 has outer threads 31 so as to be threadedly connected with the inner threads 14. Therefore, the elastic body 1 can connect with or disconnect from the reservoir 3.

According to a sixth embodiment (not shown in drawings) of the present invention, the elastic body 1 and the reservoir 3 are individually manufactured and then permanently connected to each other. The one-way device 2 may include the one-way valve 2A or the pump 2B.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. An adult toy comprising:
an elongated elastic body having an inner space defined therein and an opening defined at a first end of the elastic body; and
a sealing device disposed at the opening to seal the inner space of the elastic body, the sealing device having a path which allows fluid to flow into or out of the inner space, the path is capable of being closed to prevent the fluid in the inner space from leaking out of the inner space.

2. The adult toy as claimed in claim 1, wherein the sealing device is an elastic plug and elasticity of the sealing device makes the path of the sealing member be closed and prevents the fluid from leaking out of the inner space.

3. The adult toy as claimed in claim 1, wherein the elastic body comprises a plurality of bosses extending from an outer surface thereof.

4. An adult toy comprising:
an elongated elastic body having an inner space defined therein and an opening defined at a first end of the elastic body;
a reservoir connected to the opening of the elastic body; and
a one-way device including a switch and disposed at the opening of the elastic body to seal the inner space.

5. The adult toy as claimed in claim 4, wherein the one-way device is a one-way valve and the reservoir is a resilient reservoir.

6. The adult toy as claimed in claim 5, wherein the reservoir is an enclosed reservoir.

7. The adult toy as claimed in claim 5, wherein the reservoir includes a hole.

8. The adult toy as claimed in claim 4, wherein the one-way device includes a pump.

9. The adult toy as claimed in claim 4, wherein the reservoir is permanently connected with the elastic body.

10. The adult toy as claimed in claim 4, wherein the reservoir and the elastic body are two individual parts which are detachably connected to each other.

11. An adult toy comprising:
an elongated elastic body having an inner space defined therein and an opening defined at a first end of the elastic body;
a reservoir connected to the opening of the elastic body; and
a one-way device including a switch and disposed at an opening of the reservoir, when the reservoir is connected to the opening of the elastic body, the inner space is sealed by the one-way device.

12. The adult toy as claimed in claim 11, wherein the one-way device is a one-way valve and the reservoir is a resilient reservoir.

13. The adult toy as claimed in claim 12, wherein the reservoir is an enclosed reservoir.

14. The adult toy as claimed in claim 12, wherein the reservoir includes a hole.

15. The adult toy as claimed in claim 11, wherein the one-way device includes a pump.
